(19) United States
(12) Patent Application Publication
(10) Pub. No.: US 2021/0085197 A1
(43) Pub. Date: Mar. 25, 2021

(54) PHYSIOLOGICAL INFORMATION MEASUREMENT METHOD, PHYSIOLOGICAL INFORMATION MEASUREMENT PROGRAM, AND PHYSIOLOGICAL INFORMATION MEASUREMENT APPARATUS

(71) Applicant: NIHON KOHDEN CORPORATION, Tokyo (JP)

(72) Inventors: Yoshihiro SUGO, Tokorozawa-shi (JP); Yoshiharu HARADA, Tokorozawa-shi (JP); Mami SAKAI, Tokorozawa-shi (JP)

(21) Appl. No.: 17/026,704

(22) Filed: Sep. 21, 2020

(30) Foreign Application Priority Data
Sep. 25, 2019 (JP) 2019-173896

(51) Int. Cl. A61B 5/021; A61B 5/00; A61B 5/02

(52) U.S. Cl. CPC A61B 5/02125; A61B 5/02007; A61B 5/7235

(57) ABSTRACT

A physiological information measurement method includes the steps of: measuring a first pulse wave in a first portion of a subject; measuring a second pulse wave in a second portion of the subject; acquiring a corrected waveform data set that is based on the first and second waveforms, in which at least one of the corrected waveform data set is a waveform that is corrected at least one of the first and second waveforms based on a phase difference between a first waveform corresponding to the first pulse wave, and a second waveform corresponding to the second pulse wave; and outputting a Lissajous figure based on the corrected waveform data set.

### CROSS-REFERENCE TO RELATED APPLICATIONS

The present application claims priority from Japanese Patent Application No. 2019-173896, filed Sep. 25, 2019 the entire content of which is incorporated herein by reference.

The presently disclosed subject matter relates to a physiological information measurement method, a physiological information measurement program, and a physiological information measurement apparatus.

### BACKGROUND

JP2001-245856A discloses a vascular tone measurement apparatus that determines the systole and diastole of the blood vessel by using a Lissajous figure in which the blood pressure waveform is set on the X-axis, and the intravascular volume waveform is set on the Y-axis.

### SUMMARY

In the apparatus that is disclosed in JP2001-245856A, when a medical person is to know the vascular state such as the state of the vascular tone of the subject from a Lissajous figure, there is a case where a determination based on the skill and experience of the medical person is necessary. Therefore, there is a possibility that determinations by medical persons may be dispersed, and a case where the vascular state of the subject cannot be quantitatively evaluated may occur.

The presently disclosed subject matter is provided with a physiological information measurement method, physiological information measurement program, and physiological information measurement apparatus that can quantitatively evaluate physiological information such as the vascular tone which indicates a change of the elasticity of the blood vessel.

A first aspect of physiological information measurement method includes:

measuring a first pulse wave in a first portion of a subject;

measuring a second pulse wave in a second portion of the subject;

acquiring a corrected waveform data set that is based on the first and second waveforms, in which at least one of the corrected waveform data set is a waveform that is corrected at least one of the first and second waveforms based on a phase difference between a first waveform corresponding to the first pulse wave, and a second waveform corresponding to the second pulse wave; and

outputting a Lissajous figure based on the corrected waveform data set.

A second aspect of non-transitory computer readable medium including a physiological information measurement program is to be executed by a computer that includes at least a processor and a memory, to cause the processor to:

acquire a corrected waveform data set that is based on a first waveform corresponding to a first pulse wave in a first portion of a subject, and a second waveform corresponding to a second pulse wave in a second portion of the subject, in which at least one of the corrected waveform data set is a waveform that is corrected at least one of the first and second waveforms based on a phase difference between the first waveform and the second waveform; and

output a Lissajous figure based on the corrected waveform data set.

A third aspect of physiological information measurement apparatus includes a processor and a memory, in which the processor and the memory are configured to execute a physiological information measurement program.

The physiological information measurement program includes:
acquiring a corrected waveform data set that is based on a first waveform corresponding to a first pulse wave in a first portion of a subject, and a second waveform corresponding to a second pulse wave in a second portion of the subject, wherein at least one of the corrected waveform data set is a waveform that is corrected at least one of the first and second waveforms based on a phase difference between the first waveform and the second waveform; and
outputting a Lissajous figure based on the corrected waveform data set.

### BRIEF DESCRIPTION OF DRAWINGS

FIG. 1 illustrates a configuration example of a vascular tone measurement system of an embodiment.

FIG. 2 exemplifies a continuous waveform that is output from a blood pressure monitor.

FIG. 3 exemplifies a continuous waveform that is output from a pulse oximeter.

FIG. 4 illustrates an example of a blood pressure waveform in a region that is in the waveform of FIG. 2, and that is enclosed by a broken line.

FIG. 5 illustrates an example of an intravascular volume waveform in a region that is in the waveform of FIG. 3, and that is enclosed by a broken line.

FIG. 6 exemplifies a correlogram that is used in a correction of an intravascular volume waveform.

FIG. 7 illustrates an example of a Lissajous figure in the case where an intravascular volume waveform is corrected by an autocorrelation function.

FIG. 8 illustrates an example of a Lissajous figure in the case where an intravascular volume waveform is corrected based on a difference between pulse wave transit times.

FIG. 9 exemplifies a trend of a correlation coefficient between first and second waveforms in the case where the second waveform is not corrected.

FIG. 10 exemplifies trends of a correlation coefficient between first and second waveforms in the case where the second waveform is corrected by using an autocorrelation function, and in another case where the second waveform is corrected based on a difference between pulse wave transit times.

FIG. 11 is a flowchart exemplifying the operation of a vascular tone measurement apparatus.

### DETAILED DESCRIPTION

Hereinafter, an embodiment will be described in detail with reference to the accompanying drawings.

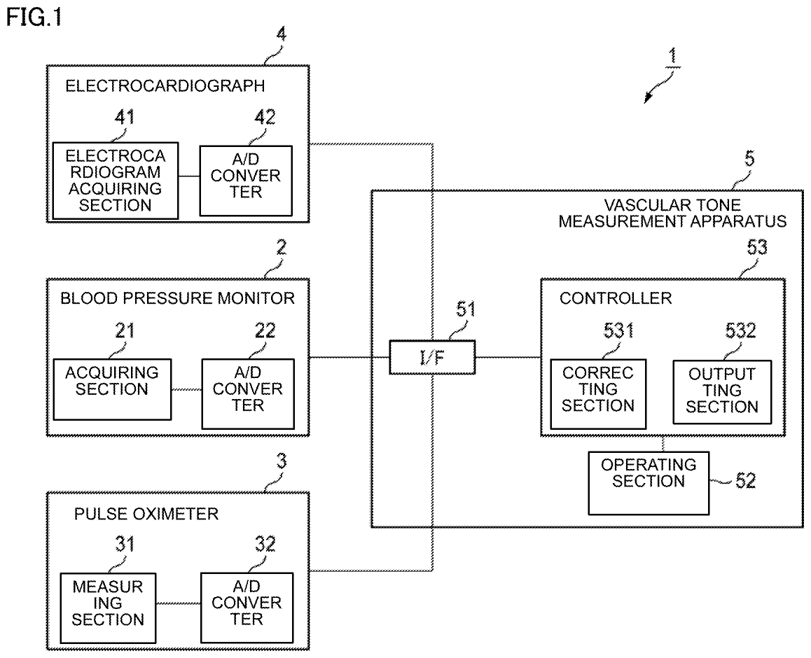

FIG. 1 illustrates a configuration example of a vascular tone measurement system (an example of a physiological information measurement system) 1 of the embodiment. The vascular tone measurement system 1 may include a blood pressure monitor 2, a pulse oximeter 3, an electrocardiograph 4, and a vascular tone measurement apparatus 5 (an example of the physiological information measurement apparatus).

First, the blood pressure monitor 2, pulse oximeter 3, and electrocardiograph 4 that are used in the vascular tone measurement system 1 will be described. The blood pressure monitor 2 may include an acquiring section 21 and an A/D converter 22. The acquiring section 21 is configured so as to be able to acquire the blood pressure (for example, the systolic blood pressure and the diastolic blood pressure) of the blood vessel of a subject, and the radial artery waveform. The acquiring section 21 may include a blood pressure sensor of, for example, the continuous measurement type. A blood pressure sensor of the continuous measurement type continuously measures the value of the blood pressure. A blood pressure sensor of the continuous measurement type continuously measures the blood pressure per beat. An example of a blood pressure sensor of the continuous measurement type is a blood pressure sensor of the tonometry type or the optical type.

In the tonometry type, a pressure sensor is directly contacted with a living body portion such as the wrist through which an artery such as the radial artery passes, and the blood pressure value is measured by using information detected by the pressure sensor. In the optical type, a blood vessel is impinged by a light beam, and the blood pressure value is measured from a reflected or transmitted light beam of the light beam. The acquiring section 21 is attached to any portion of the body of the subject. In the specification, the portion to which the acquiring section 21 is attached is referred to as the first portion. For example, the first portion is the wrist. The acquiring section 21 measures the blood pressure pulse wave (an example of the first pulse wave), and outputs the measured blood pressure pulse wave to the A/D converter 22.

The A/D converter 22 coverts the blood pressure pulse wave that is output from the acquiring section 21, to a digital signal at every predetermined sampling period. The digital signal is transmitted as a blood pressure waveform (an example of the first waveform) corresponding to the blood pressure pulse wave, to the vascular tone measurement apparatus 5.

The pulse oximeter 3 may include a measuring section 31 and an A/D converter 32. The pulse oximeter 3 emits a light beam to the finger or the like of the subject, and measures the intensity of the transmitted or reflected light beam. A waveform that is obtained from a change over time of the intensity of the transmitted light beam is a photoplethysmogram (an example of the second pulse wave). The pulse oximeter 3 is a device that performs a calculation on a photoplethysmogram that is obtained by emission of light beams of a plurality of wavelengths, to take out a light absorption characteristics of only the arterial blood, and that outputs the arterial oxygen saturation (SpO₂). The output waveform is the intravascular volume waveform (an example of the second waveform) corresponding to the artery pulse wave.

The measuring section 31 may include a probe that can be attached to, for example, the finger of the subject. The measuring section 31 is attached to any portion that is in the body of the subject, and that is different from the first portion. In the specification, the portion to which the measuring section 31 is attached is referred to as the second portion. For example, the second portion is the fingertip. The measuring section 31 is configured so as to output the oxygen saturation in the blood vessel of the subject, by using a detection signal of a transmitted or reflected light beam that is supplied from a light detector which is provided in the probe.

The A/D converter 32 converts the photoplethysmogram that is supplied from the measuring section 31, to a digital signal at every predetermined sampling period. The digital signal is transmitted to the vascular tone measurement apparatus 5.

The arterial vessel is an elastic vessel that has a muscle tissue in the outer circumference, and therefore expands or contracts depending on the internal pressure. The base line of the waveform of the digital signal that is output from the pulse oximeter 3 corresponds to the amount of the arterial blood in the case where the arterial pressure does not exist (the diastolic blood pressure state). By contrast, the wave crest portion of the waveform corresponds to the increased blood amount in a time period when the pulsation changes while including the amount of the arterial blood in the case where the arterial pressure is maximum (the systolic blood pressure state). Therefore, the continuous waveform that is output by the pulse oximeter 3 indicates a change amount that is a value relative to the intravascular volume of the artery. Consequently, the digital signal can be handled as an intravascular volume waveform that corresponds to the photoplethysmogram.

The electrocardiograph 4 may include an electrocardiogram acquiring section 41 and an A/D converter 42. The electrocardiogram acquiring section 41 is configured so as to be able to acquire an electrocardiogram waveform of the subject. The electrocardiogram acquiring section 41 may include, for example, electrodes that can be attached to the subject. The electrocardiogram acquiring section 41 acquires electrocardiogram information of the subject based on measured potentials that are supplied from the electrodes attached to the subject. The electrocardiogram acquiring section 41 outputs an electrocardiogram waveform (analog signal) based on the acquired electrocardiogram information.

The A/D converter 42 converts the electrocardiogram waveform to a digital signal at every predetermined sampling period. The digital signal is transmitted to the vascular tone measurement apparatus 5.

The vascular tone measurement apparatus 5 may include a communication interface (I/F) 51, an operating section 52, and a controller 53.

The communication interface 51 enables connections to the blood pressure monitor 2, the pulse oximeter 3, and the electrocardiograph 4. The vascular tone measurement apparatus 5 can appropriately communicate with the blood pressure monitor 2, the pulse oximeter 3, and the electrocardiograph 4 through the communication interface 51. The blood pressure monitor 2, the pulse oximeter 3, and the electrocardiograph 4 may have a configuration in which they are modularized and integrated with the vascular tone measurement apparatus 5.

The operating section 52 is configured so as to receive an input operation performed by an operator who operates the vascular tone measurement apparatus 5, and produce an instruction signal corresponding to the input operation. The operating section 52 is configured by, for example, a touch panel that is overlappingly placed on a displaying section of the vascular tone measurement apparatus 5, or operation buttons that are disposed on the case of the vascular tone measurement apparatus 5.

The controller 53 may include at least one memory and at least one processor. The memory is configured by, for example, a Read Only Memory (ROM) in which various programs and the like are stored, and a Random Access Memory (RAM) having a plurality of work areas in which various programs to be executed by the processor, and the like are stored. For example, the processor is a Central Processing Unit (CPU), and configured so as to load designated ones of the various programs incorporated in the ROM, into the RAM, and execute various processes in cooperation with the RAM.

The controller 53 may include a correcting section 531 and an outputting section 532. Various functions including the functions of the correcting section 531 and the outputting section 532 are realized by execution of a vascular tone measurement program (an example of the physiological information measurement program) stored in the memory of the controller 53, by the processor. Alternatively, various functions may be realized by electronic circuits that are hardware, or at least a part of the functions may be realized by software, i.e., processes that are executed on a computer.

Based on the phase difference between the blood pressure waveform corresponding to the blood pressure pulse wave, and the intravascular volume waveform corresponding to the photoplethysmogram, the correcting section 531 corrects the blood pressure waveform and the intravascular volume waveform, or the blood pressure waveform or the intravascular volume waveform. For example, the blood pressure waveform and the intravascular volume waveform, or the blood pressure waveform or the intravascular volume waveform may be corrected by using an autocorrelation function, or corrected based on the difference between the pulse wave transit time of the blood pressure waveform and that of the intravascular volume waveform. The pulse wave transit time (which will be hereinafter abbreviated to PWTT) means a time interval between a peak point of an R wave and a rise point of a pulse waveform appearing due to the R wave. In the case where the intravascular volume waveform is to be corrected based on the difference between the PWTTs of the blood pressure waveform and the intravascular volume waveform, therefore, the correcting section 531 requires electrocardiogram information. In this case, that is, the vascular tone measurement system 1 includes the electrocardiograph 4. In the case where the rise point of the pulse wave is to be detected in each of the blood pressure pulse wave and the intravascular volume waveform, and a correction is to be performed so that the rise points coincide with each other, an electrocardiogram may not be included.

In the specification, a term “corrected waveform data set” may be used for describing a data set that is acquired by correcting at least one of the first waveform and the second waveform. The corrected waveform data set is one of: a data set containing the blood pressure pulse wave that is corrected by, for example, the correcting section 531, and the intravascular volume waveform that is corrected by the correcting section 531; that containing the blood pressure pulse wave that is corrected by the correcting section 531, and the intravascular volume waveform that is not corrected; and that containing the blood pressure pulse wave that is not corrected, and the intravascular volume waveform that is corrected by the correcting section 531.

The outputting section 532 is configured so as to produce a Lissajous figure based on the corrected waveform data set, and output the Lissajous figure to the displaying section of the vascular tone measurement apparatus 5. Alternatively, the outputting section 532 may output the Lissajous figure on a paper sheet. Moreover, the outputting section 532 may output the Lissajous figure to an external apparatus that is wiredly or wirelessly connected with the vascular tone measurement apparatus 5.

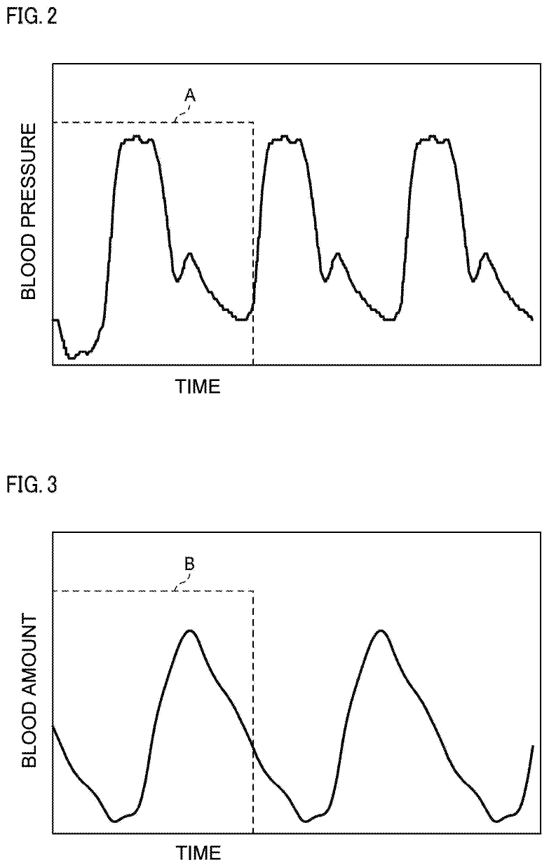

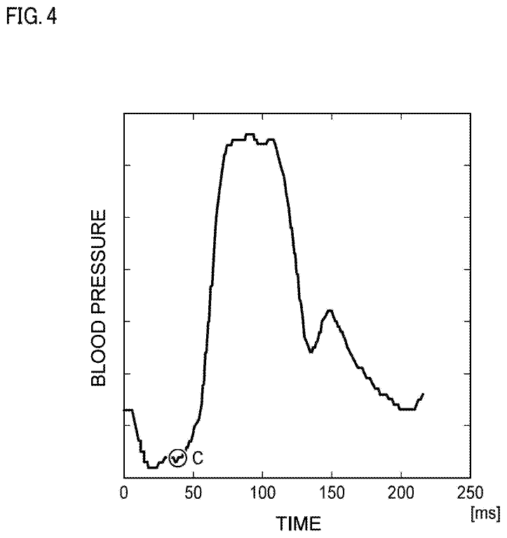

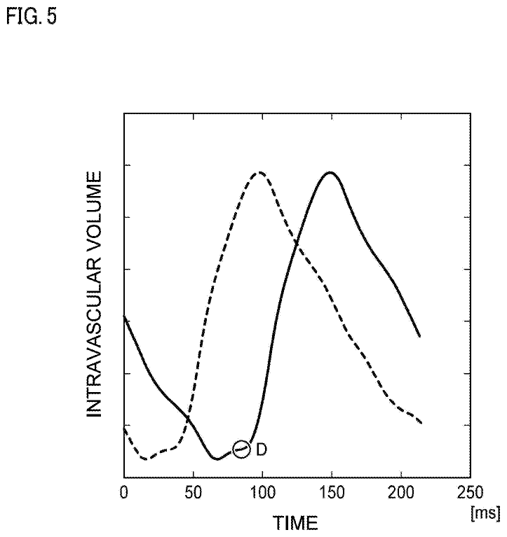

FIG. 2 exemplifies the continuous waveform that is output from the blood pressure monitor 2. FIG. 3 exemplifies the continuous waveform that is output from the pulse oximeter 2. FIG. 4 illustrates an example of the blood pressure waveform in a region A that is in the waveform of FIG. 2, and that is enclosed by the broken line, and FIG. 5 illustrates an example of the intravascular volume waveform in a region B that is in the waveform of FIG. 3, and that is enclosed by the broken line.

The waveforms that are contained in the region A of FIG. 2, and the region B of FIG. 3 are waveforms per unit period, respectively. That is, the blood pressure waveform per unit period is the waveform that is exemplified in FIG. 4, and the intravascular volume waveform per unit period is the waveform that is exemplified in FIG. 5, and that is indicated by the solid line.

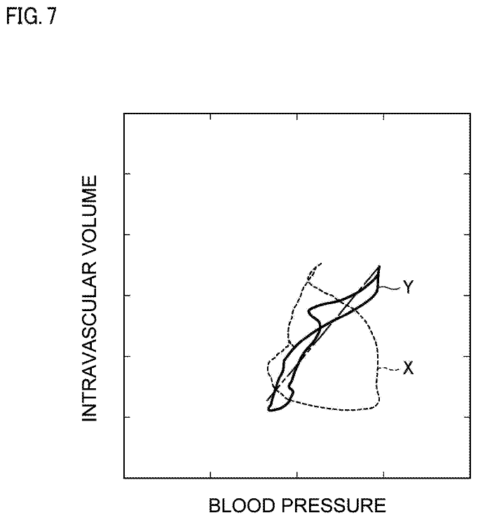

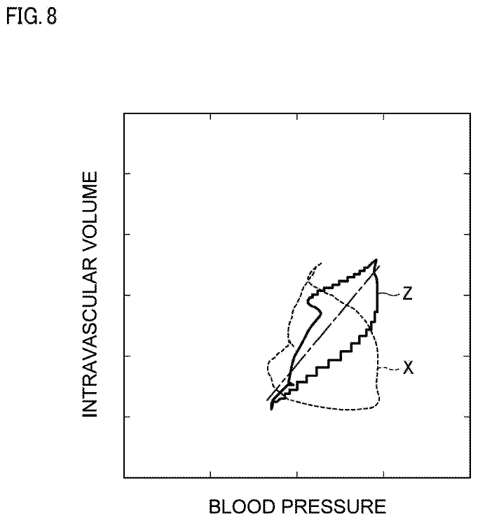

When a Lissajous figure for measuring the vascular tone is output based on the waveform exemplified in FIG. 4, and that indicated by the solid line in the waveforms exemplified in FIG. 5, the output Lissajous figure has a wide shape that biaxially extends as exemplified in the Lissajous figure X of FIGS. 7 and 8. In order to correctly evaluate the vascular tone, the medical person tries to recognize, from the output Lissajous figure, the slope (the inclination that is obtained by a regression analysis for providing the Lissajous figure with an approximate line) of the Lissajous figure. When the Lissajous figure has a wide shape that biaxially extends, however, the medical person hardly recognizes the slope, and there is a case where it is difficult to quantitatively know the vascular tone based on the slope. Therefore, the inventors have studied the cause of the formation of a wide Lissajous figure that biaxially extends. Then, the inventors have noticed that, not only the shapes of the blood pressure waveform and the intravascular volume waveform, but also the phase difference between the blood pressure waveform and the intravascular volume waveform affects the shape of the Lissajous figure that is output from the outputting section 532.

As described above, the portion (first portion) to which the acquiring section 21 of the blood pressure monitor 2 is attached, and that (second portion) to which the measuring section 31 of the pulse oximeter 3 is attached are different from each other. That is, the distance from the heart to the first portion is different from that from the heart to the second portion. The inventors have noticed that the difference in positions of the measurement portions causes a phase difference to occur between the blood pressure waveform and the intravascular volume waveform. As exemplified in FIGS. 4 and 5, the rising time (the point C in FIG. 4) of the blood pressure waveform is 40 milliseconds, and that (the point D in FIG. 5) of the intravascular volume waveform is 90 milliseconds. Namely, a phase difference of 50 milliseconds exists between the blood pressure waveform and the intravascular volume waveform. The inventors have arrived at the thought that, in the case where the vascular state of the subject is to be determined based on a Lissajous figure, the width of the output Lissajous figure can be reduced by correcting the phase difference, with the result that the determination of the vascular state of the subject seems to be easily performed.

Based on the result of the study, the vascular tone measurement apparatus 5 of the embodiment is configured so as to include the correcting section 531. The correcting section 531 calculates the phase difference that occurs between the blood pressure waveform and the intravascular volume waveform, and corrects the intravascular volume waveform based on the phase difference. Examples of the method of correcting the intravascular volume waveform are a correction using an autocorrelation function, and that based on the difference of PWTTs.

In the case where the intravascular volume waveform is to be corrected by using an autocorrelation function, the correcting section 531 calculates an autocorrelation coefficient based on the intravascular volume waveform. The autocorrelation coefficient indicates the strength of the correlation between the acquired intravascular volume waveform, and the intravascular volume waveform that is corrected by the correcting section 531. The autocorrelation coefficient is −1 or more and 1 or less. When the autocorrelation coefficient is 1, the acquired intravascular volume waveform and the corrected intravascular volume waveform are coincident with each other in viewpoints of period, amplitude, and phase. On the other hand, the larger the difference in the viewpoints of period, amplitude, and phase between the acquired intravascular volume waveform and the corrected intravascular volume waveform, the smaller the autocorrelation coefficient.

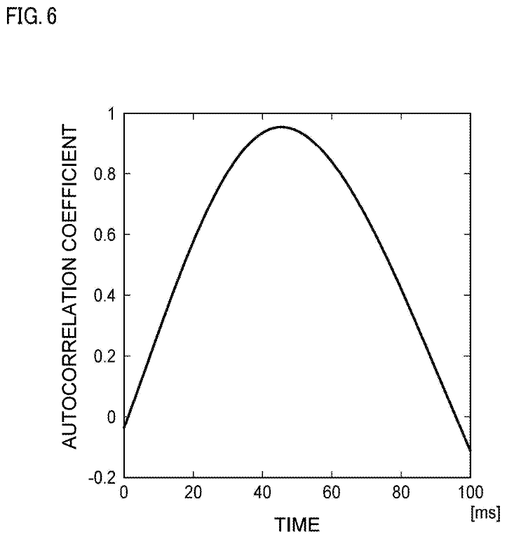

A method of calculating the autocorrelation coefficient will be specifically described with reference to FIG. 6. The correcting section 531 moves the intravascular volume waveform toward the origin in parallel to the time axis to calculate the autocorrelation coefficient. In the embodiment, as exemplified in FIG. 6, the autocorrelation coefficient is maximum when the intravascular volume waveform is moved toward the origin by 45 milliseconds in parallel to the time axis. Therefore, the correcting section 531 performs a correction in which the intravascular volume waveform is moved toward the origin by 45 milliseconds in parallel to the time axis. The corrected intravascular volume waveform is indicated by the broken line in FIG. 5. The information relating to the corrected intravascular volume waveform is stored in the controller 53.

Alternatively, the intravascular volume waveform may be corrected based on the difference between the PWTTs of the blood pressure waveform and the intravascular volume waveform. In the alternative, the correcting section 531 calculates the difference between the PWTTs of the blood pressure waveform and the intravascular volume waveform from the rising time (the point C in FIG. 4) of the blood pressure waveform, and that (the point D in FIG. 5) of the intravascular volume waveform. In the embodiment, the difference between the rising times of the blood pressure waveform and the intravascular volume waveform, i.e., that between the PWTTs of the blood pressure waveform and the intravascular volume waveform in is 50 milliseconds. The correcting section 531 performs a correction in which the intravascular volume waveform is moved toward the origin by the calculated difference (i.e., 50 milliseconds) between the PWTTs, in parallel to the time axis.

FIG. 7 exemplifies a Lissajous figure in the case where the intravascular volume waveform is corrected by an autocorrelation function, and FIG. 8 exemplifies a Lissajous figure in the case where the intravascular volume waveform is corrected based on the difference between the PWTTs. In FIGS. 7 and 8, each of the shapes indicated by the broken lines illustrates a Lissajous figure X that is output from the outputting section 532 in the case where the intravascular volume waveform is not corrected. The Lissajous figure X is output based on the waveform exemplified in FIG. 4, and the waveform that is exemplified in FIG. 5, and that is indicated by the solid line. In FIG. 7, the shape indicated by the thick line illustrates a Lissajous figure Y that is output from the outputting section 532 in the case where the intravascular volume waveform is corrected by an autocorrelation function. The Lissajous figure Y is output based on the waveform exemplified in FIG. 4, and the waveform that is exemplified in FIG. 5, and that is indicated by the broken line. In FIG. 8, the shape indicated by the thick line illustrates a Lissajous figure Z that is output from the outputting section 532 in the case where the intravascular volume waveform is corrected based on the difference between the PWTTs of the blood pressure waveform and the intravascular volume waveform. The Lissajous figure Z is output based on the waveform exemplified in FIG. 4, and that obtained by correcting the waveform that is exemplified in FIG. 5, and that is indicated by the solid line, based on the difference between the PWTTs of the blood pressure waveform and the intravascular volume waveform. In FIG. 7, the straight line indicated by the dash-dot line indicates the slope in the case where the intravascular volume waveform is corrected by the autocorrelation function. In FIG. 8, the straight line indicated by the dash-dot line indicates the slope in the case where the intravascular volume waveform is corrected based on the difference between the PWTTs of the blood pressure waveform and the intravascular volume waveform. For example, these slopes may be produced by performing a statistical process such as a regression analysis for providing the Lissajous figure with an approximate line, on the blood pressure waveform and the corrected intravascular volume waveform. The lager the inclination of the slope, the higher the degree of correlation between the blood pressure waveform and the corrected intravascular volume waveform.

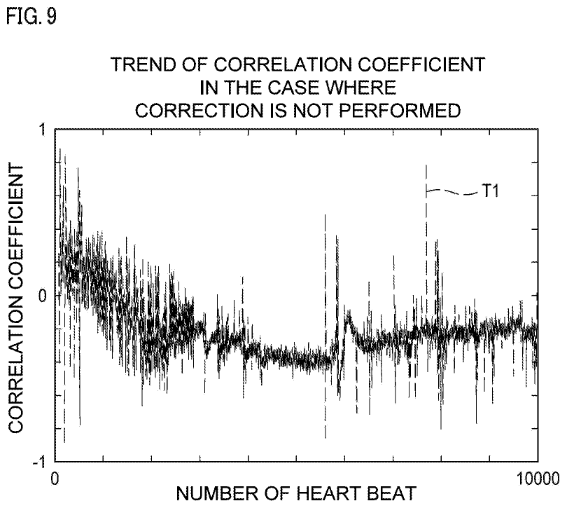

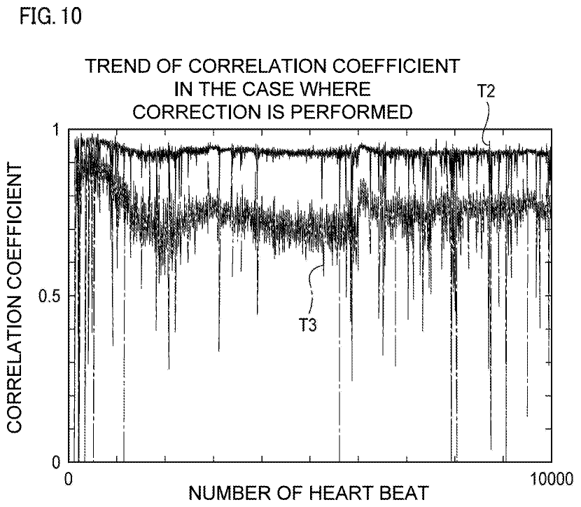

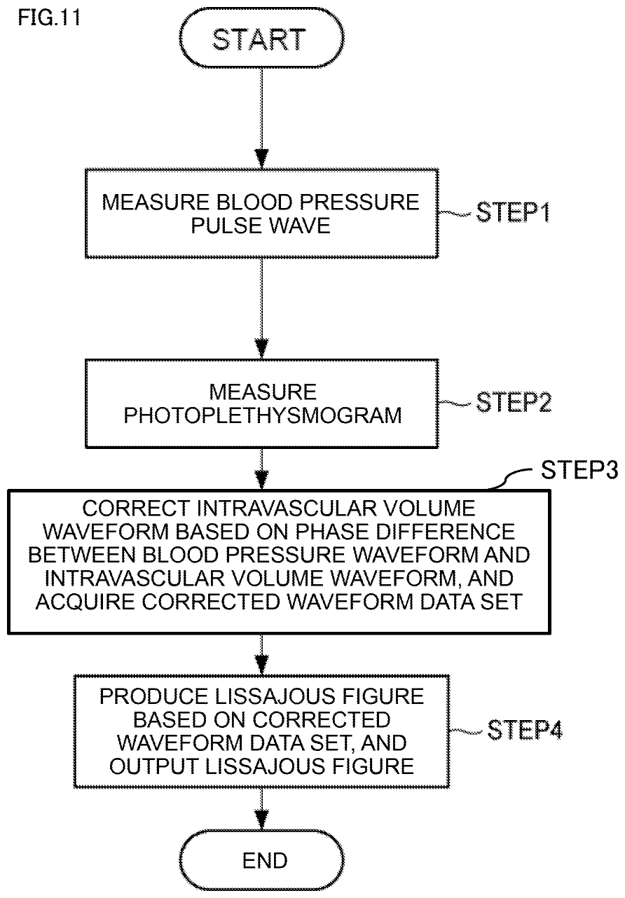

FIG. 9 exemplifies a trend T1 of the correlation coefficient between the blood pressure waveform and the intravascular volume waveform in the case where the intravascular volume waveform is not corrected, and FIG. 10 exemplifies a trend T2 of the correlation coefficient between the blood pressure waveform and the intravascular volume waveform in the case where the intravascular volume waveform is corrected by using an autocorrelation function, and a trend T3 of the correlation coefficient between the blood pressure waveform and the intravascular volume waveform in the case where the intravascular volume waveform is corrected based on the difference between the PWTTs. In the case where the intravascular volume waveform is not corrected, as exemplified in FIG. 9, the correlation coefficient between the blood pressure waveform and the intravascular volume waveform has a value that is remote from 1, and that is close to 0 or −1. In the case where the intravascular volume waveform is corrected, by contrast, the correlation coefficient between the blood pressure waveform and the intravascular volume waveform has a value that is close to 1 as exemplified in FIG. 10. The closer the value of the correlation coefficient between the blood pressure waveform and the intravascular volume waveform is to 1, the smaller the divergence between the blood pressure waveform and the intravascular volume waveform in period, amplitude, and phase, and the more similar the Lissajous figure is to a linear shape. By contrast, the smaller the correlation coefficient between the blood pressure waveform and the intravascular volume waveform (that is, the remoter the value of the correlation coefficient from 1), the larger the divergence between the blood pressure waveform and the intravascular volume waveform in period, amplitude, and phase, and the Lissajous figure has a wide shape that biaxially extends.

As exemplified in FIGS. 7 and 8, the Lissajous figure X has a wide shape that biaxially extends, as compared with the Lissajous figures Y and Z. As exemplified in FIGS. 9 and 10, moreover, the correlation coefficient between the blood pressure waveform and the intravascular volume waveform in the case where the intravascular volume waveform is not corrected is smaller than that between the blood pressure waveform and the intravascular volume waveform in the case where the intravascular volume waveform is corrected. In other words, the correlation coefficient between the blood pressure waveform and the intravascular volume waveform in the case where the intravascular volume waveform is corrected has a value that is closer to 1 than the value of the correlation coefficient between the blood pressure waveform and the intravascular volume waveform in the case where the intravascular volume waveform is not corrected. Therefore, the smaller the correlation coefficient between the blood pressure waveform and the intravascular volume waveform, the wider the Lissajous figure has a shape that biaxially extends. By contrast, the closer the value of the correlation coefficient between the blood pressure waveform and the intravascular volume waveform is to 1, the narrower the width of the Lissajous figure in the biaxial directions. When the width of a Lissajous figure is narrow in the biaxial directions, the Lissajous figure becomes close to a straight line, and therefore the medical person easily recognizes the correct slope of the Lissajous figure based on the Lissajous figure.

As exemplified in FIGS. 7 and 8, when the Lissajous figures Y and Z are compared with each other, moreover, the width of the Lissajous figure Z is larger in the biaxial directions than that of the Lissajous figure Y. As exemplified in FIG. 10, furthermore, the correlation coefficient between the blood pressure waveform and the intravascular volume waveform in the case where the intravascular volume waveform is corrected by using an autocorrelation coefficient is closer to 1 than that between the blood pressure waveform and the intravascular volume waveform in the case where the intravascular volume waveform is corrected based on the difference between the PWTTs of the blood pressure waveform and the intravascular volume waveform.

FIG. 11 is a flowchart exemplifying the operation of the vascular tone measurement apparatus 5, i.e., a vascular tone measurement method (an example of the physiological information measurement method). The operation of the flowchart is executed by the controller 53 in accordance with the vascular tone measurement program stored in the memory of the controller 53.

The controller 53 controls the blood pressure monitor 2 so as to measure the blood pressure pulse wave in the wrist of the subject, and transmit a blood pressure waveform corresponding to the measured blood pressure pulse wave to the vascular tone measurement apparatus 5 (STEP 1).

The controller 53 controls the pulse oximeter 3 so as to measure the photoplethysmogram in the fingertip of the subject, and transmit an intravascular volume waveform corresponding to the measured photoplethysmogram to the vascular tone measurement apparatus 5 (STEP 2).

The controller 53 calculates the phase difference between the blood pressure waveform corresponding to the blood pressure pulse wave, and the intravascular volume waveform corresponding to the photoplethysmogram. The controller 53 corrects the intravascular volume waveform based on the calculated phase difference, and acquires a corrected waveform data set containing the corrected intravascular volume waveform and the blood pressure waveform (STEP 3).

The controller 53 produces a Lissajous figure based on the corrected waveform data set, and causes the Lissajous figure to be displayed on the displaying section of the vascular tone measurement apparatus 5 (STEP 4).

According to the above-described configuration, based on the phase difference between the first waveform corresponding to the first pulse wave, and the second waveform corresponding to the second pulse wave, at least one of the first and second waveforms is corrected to acquire the corrected waveform data set that is based on the first and second waveforms. Then, a Lissajous figure is output based on the acquired corrected waveform data set. Even when a medical person does not perform determination based on the skill and experience, therefore, the medical person can quantitatively evaluate physiological information such as the vascular tone of the subject from the output Lissajous figure.

According to the configuration, the controller 53 of the vascular tone measurement apparatus 5 corrects the intravascular volume waveform based on the phase difference between the blood pressure waveform (first waveform) and the intravascular volume waveform (second waveform), acquires the corrected waveform data set, and then outputs a Lissajous figure based on the corrected waveform data set. In the output Lissajous figure, the width is not wide in the biaxial directions, and therefore the medical person can easily recognize the slope of the Lissajous figure. When the vascular state of the subject is to be known from a Lissajous figure, consequently, the medical person is not required to estimate the slope of the Lissajous figure based on the skill and experience. As a result, the medical person can quantitatively evaluate the vascular tone.

According to the configuration, moreover, the intravascular volume waveform is corrected by using an autocorrelation function. When the vicinity of the rising of the blood pressure waveform or the intravascular volume waveform is contaminated with artifacts (noises), or has a peculiar shape, the reliabilities of the measurements of the blood pressure waveform and the intravascular volume waveform are lowered. According to the thus configured vascular tone measurement apparatus 5, however, the phase difference between the blood pressure waveform and the intravascular volume waveform is calculated without depending on the reliabilities of the measurements of the blood pressure waveform and the intravascular volume waveform, and the intravascular volume waveform can be corrected based on the phase difference.

According to the configuration, furthermore, the intravascular volume waveform is corrected based on the difference between the PWTTs of the blood pressure waveform and the intravascular volume waveform that are used also as diagnosis information, and that are known as standard values or the like. As a result, the outputting section 532 of the vascular tone measurement apparatus 5 can output a Lissajous figure from which the medical person can quantitatively evaluate the vascular tone.

The presently disclosed subject matter is not limited to the above-described embodiment, and may be freely subjected to modifications, improvements, and the like. In addition, the materials, shapes, dimensions, values, forms, numbers, installation places, and the like of the components of the above-described embodiment are arbitrary and not limited insofar as the presently disclosed subject matter can be achieved.

Although, in the embodiment, the intravascular volume waveform is corrected, the blood pressure waveform may be corrected, or both the blood pressure waveform and the intravascular volume waveform may be corrected.

Although, in the embodiment, the first waveform is the blood pressure waveform, and the second waveform is the intravascular volume waveform, the first and second waveforms may be physiological waveforms relating to other physiological information.

Although, in the embodiment, STEPS 1 and 2 exemplified in FIG. 11 are preferably executed at the same time, these steps may be individually executed. At this time, either of STEPS 1 and 2 may be first executed.

What is claimed is:

1. A physiological information measurement method comprising:
measuring a first pulse wave in a first portion of a subject;
measuring a second pulse wave in a second portion of the subject;
acquiring a corrected waveform data set that is based on the first and second waveforms, wherein at least one of the corrected waveform data set is a waveform that is corrected at least one of the first and second waveforms based on a phase difference between a first waveform corresponding to the first pulse wave, and a second waveform corresponding to the second pulse wave; and
outputting a Lissajous figure based on the corrected waveform data set.

2. The physiological information measurement method according to claim 1, wherein the acquiring includes correction by using an autocorrelation function.

3. The physiological information measurement method according to claim 1, wherein the acquiring includes correction based on a difference between a pulse wave transit time of the first waveform and a pulse wave transit time of the second waveform.

4. A non-transitory computer readable medium including a physiological information measurement program that is to be executed by a computer that includes at least a processor and a memory, to cause the processor to:
acquire a corrected waveform data set that is based on a first waveform corresponding to a first pulse wave in a first portion of a subject, and a second waveform corresponding to a second pulse wave in a second portion of the subject, wherein at least one of the corrected waveform data set is a waveform that is corrected at least one of the first and second waveforms based on a phase difference between to the first waveform and the second waveform; and
output a Lissajous figure based on the corrected waveform data set.

5. The non-transitory computer readable medium according to claim 4, wherein the program causes the processor to correct by using an autocorrelation function.

6. The non-transitory computer readable medium according to claim 4, wherein the program causes the processor to correct based on a difference between a pulse wave transit time of the first waveform and a pulse wave transit time of the second waveform.

7. A physiological information measurement apparatus comprising a processor and a memory,
wherein the processor and the memory are configured to execute a physiological information measurement program that comprises:
acquiring a corrected waveform data set that is based on a first waveform corresponding to a first pulse wave in a first portion of a subject, and a second waveform corresponding to a second pulse wave in a second portion of the subject, wherein at least one of the corrected waveform data set is a waveform that is corrected at least one of the first and second waveforms based on a phase difference between the first waveform and the second waveform; and
outputting a Lissajous figure based on the corrected waveform data set.
